(19) United States
(12) Patent Application Publication
(10) Pub. No.: US 2026/0355124 A1
(43) Pub. Date: Nov. 12, 2026

(54) METHOD FOR PREPARING EXTRACELLULAR VESICLES HAVING REDUCED NON-SPECIFIC BINDING/ENDOCYTOSIS

(71) Applicant: PANEXO BIOTECH CO., LTD, Beijing (CN)

(72) Inventors: Yi WANG, Beijing (CN); Jialei YANG, Beijing (CN); Haifeng SONG, Beijing (CN); Jing ZHANG, Beijing (CN); Yanan DONG, Beijing (CN); Jun LI, Beijing (CN); Miaomiao XUE, Beijing (CN)

(21) Appl. No.: 19/119,462

(22) PCT Filed: Oct. 11, 2022
(86) PCT No.: PCT/CN2022/124603

§ 371 Date: Apr. 9, 2025

(51) Int. Cl. A61K 9/51; A61K 9/50; A61K 38/20; A61P 35/00; C12N 5/00; C12N 5/071; C12N 9/22; C12N 15/11; C12N 15/85; C12Q 1/26; G01N 33/50; G01N 33/569; G01N 33/573

(52) U.S. Cl. CPC A61K 9/5192; A61K 9/5068; A61K 38/208; A61P 35/00; C12N 5/0018; C12N 5/0081; C12N 5/0686; C12N 9/226; C12N 15/111; C12N 15/85; C12Q 1/26; G01N 33/5023; G01N 33/5044; G01N 33/56966; G01N 33/573; C12N 2310/20; C12N 2501/585; C12N 2510/02; G01N 2333/70596; G01N 2333/90203; G01N 2333/90241

(57) ABSTRACT

The present disclosure relates to a method for preparing extracellular vesicles with reduced non-specific binding/endocytosis. By means of performing knockout and deglycosylation operations on extracellular vesicle-associated adhesion molecules, extracellular vesicles with reduced non-specific binding/endocytosis are obtained, and the circulation time thereof in the blood is pro-longed. Furthermore, an IL-12 combined antibody is loaded onto the extracellular vesicles with reduced non-specific binding/endocytosis to achieve enhanced efficacy of IL-12 in a “multimer” mode, which further achieves targeting by means of the antibody, reducing an initial concentration of IL-12 and improving safety while also achieving efficient inhibition of tumor growth at a certain level of safety.

### CROSS-REFERENCE TO RELATED APPLICATIONS

This application is a U.S. National Stage Entry of International Patent Application No. PCT/CN2022/124603, filed Oct. 11, 2022, which is incorporated herein by reference in its entirety.

### REFERENCE TO AN ELECTRONIC SEQUENCE LISTING

This application contains references to nucleic acid sequences and/or amino acid sequences which have been submitted concurrently herewith as the sequence listing XML file entitled “82098-000013-US-NP_3_Apr_2025_ST26.xml”, file size 11,998 Bytes (B), created on 3 Apr. 2025. The aforementioned sequence listing is hereby incorporated by reference in its entirety.

### FIELD

The present disclosure relates to the technical field of modification of extracellular vesicles including exosomes, specifically relates to the knockout and deglycosylation of extracellular vesicle-associated adhesion molecules, as well as the co-loading of IL12 and an antibody into extracellular vesicles with reduced non-specific binding/endocytosis.

### BACKGROUND

Extracellular vesicles (EVs) have a vesicular structure and are secreted during cell growth. They often carry intracellular proteins and nucleic acids during their formation and carry extracellular proteins on or outside of the vesicle membrane, similar to the cell membrane. The variety of proteins on the membrane of extracellular vesicles is so great that extracellular vesicles can bind to and be endocytosed by almost all cells to varying degrees.

Cancer treatment typically combines chemotherapy, radiotherapy, surgery, and targeted therapy. Targeted therapy is a treatment method that specifically targets known cancer-causing sites at the molecular level. Corresponding therapeutic drugs can be designed to enter the body and selectively interact with these cancer-causing sites, leading to the specific death of tumor cells. Extracellular vesicles are commonly used as efficient drug delivery systems. However, their intercellular communication function can lead to unnecessary non-specific binding/endocytosis by cells, which accelerates their being cleared. Therefore, ensuring that extracellular vesicles can stably deliver drugs in vivo without being cleared is a significant technical challenge in the field.

Interleukin-12 (IL-12) is a type of cytokine and bioactive IL-12 can activate the generation of inflammatory cells. Proper activation of these cells can help to clear tumor cells, but excessive activation may cause serious side effects, such as cytokine release syndrome (CRS). Low concentrations of IL-12 lack sufficient activity to activate the immune system and then promote the clearance of tumor cells. Therefore, new methods are needed to activate the immune system effectively within a safe concentration range. In addition, enhancing the circulation time of extracellular vesicles in the bloods to ensure they effectively reach target lesions is also an urgent challenge in the field for their use as drug delivery vehicles.

### SUMMARY

In order to extend the circulation time of extracellular vesicles (EVs) in the blood and ensure their effective delivery to target lesions, the present disclosure knocks out adhesion molecules associated with EVs and performs deglycosylation to obtain EVs with reduced non-specific binding/endocytosis, thereby extending their circulation time in the blood. Further, the present disclosure co-loads IL-12 and an antibody into the EVs with reduced non-specific binding/endocytosis, enhancing the efficacy of IL-12 in a “polymeric” mode by means of receptor sequestration feature of EVs, and reducing the required concentration of IL-12 through the antibody's targeted delivery. This improves safety and at the same time achieves adequate inhibition of tumor growth.

The present disclosure provides a method for preparing an extracellular vesicle (EV) with reduced non-specific binding/endocytosis, comprising steps of:
(1) knocking out ITGB1 in HEK293F cells at the cellular level to achieve ITGB1 knockout at the EV level,
(2) sorting and verifying an ITGB1 knockout HEK293F cell line,
(3) loading NanoLuc® into EVs from the ITGB1 knockout HEK293F cell line,
(4) verifying changes in binding/endocytosis function of EVs from ITGB1 knockout HEK293F cell line at the cellular level, and
(5) verifying the effect of ITGB1 knockout on the tissue distribution of EVs in major organs in vivo.

In the present disclosure, step (1) comprises transiently transfecting ITGB1 CRISPR/Cas9/gRNA plasmid into HEK293F suspension cells using PEI transfection reagent, incubating the cells with APC anti-human ITGB1 antibody after 72 h, sorting out APC negative cell population by flow cytometry, and expanding the sorted cell population, resulting in an ITGB1 knockout HEK293F cell line.

In the present disclosure, step (2) further comprises identifying changes in ITGB1 expression on ITGB1 EVs by Western Blot.

In the present disclosure, step (3) comprises constructing pFc-TMD (NPTN-TMD)-ICD (EWI-F-ICD)-NanoLuc® plasmid using VB220306-1137jmq as a vector, co-transfecting the plasmid into the cells with Sleeping Beauty transposase expression vector pCMV-(CAT) T7-SB100X, adding Zeocin™ antibiotic at a final concentration of 100 μg/ml after 72 h until all cells in a non-stably transfected group die, expanding the cells, centrifuging cells at 5000 rpm for 30 min to collect supernatant, purifying EVs from the supernatant, establishing a standard curve of EV particle concentration-NanoLuc® enzyme activity to verify NanoLuc® activity in EVs, and treating EVs with Triton™ X-100 and Proteinase K to verify the loading position of NanoLuc® in EVs.

In the present disclosure, step (4) comprises inoculating recipient cells at 0.5E+5 cells per well into a 24-well plate, adding 50 μL of NanoLuc® detection substrate after treatment, and measuring NanoLuc® activity.

In the present disclosure, step (5) comprises injecting EVs via the tail vein at a dose of 5E+10 particles per mouse, collecting plasma from peripheral blood at 5, 15, 30, 60, 120 and 240 min, homogenizing liver, spleen, lung and kidney tissues to obtain tissue homogenates, measuring NanoLuc® enzyme activity in the plasma and tissue homogenates, establishing a standard curve of EV particle concentration-NanoLuc® enzyme activity, and calculating EV particle concentration in the tissues.

The method of the present disclosure further comprises step (6) of subjecting EVs from the ITGB1 knockout cells to deglycosylation treatment.

In the present disclosure, step (6) comprises adding 10 μl of PNGase F and 100 μl of 10× Reaction Buffer per 2E+10 EVs to EVs from the ITGB1 knockout cells, supplementing the system with PBS to 1 ml with EV concentration of 2E+10 p/ml, performing incubation at 37 °C for 1 h, and detecting CD63 and GAPDH by Western Blot.

In the method of the present disclosure, verification of changes in binding/endocytosis of EVs with recipient cells after deglycosylation at the cellular level comprises adding NanoLuc® detection substrate and measuring NanoLuc® activity.

In the method of the present disclosure, verification of the effect of deglycosylation on the tissue distribution of EVs in major organs in vivo comprises injecting EVs via the tail vein at a dose of 5E+10 particles per mouse, collecting plasma from peripheral blood at 5, 15, 30, 60, 120 and 240 min, homogenizing liver, spleen, lung and kidney tissues to obtain tissue homogenates, measuring NanoLuc® enzyme activity in the plasma and tissue homogenates, establishing a standard curve of EV particle concentration-NanoLuc® enzyme activity, and calculating EV particle concentration in the tissues.

The method of the present disclosure further comprises step (7) of loading an antibody into EVs to confer targeting function.

In the present disclosure, step (7) comprises constructing plgkappa-HA-HN3-TMD (PDGFRB) plasmid using pHEK293_Ultra_Expression_I (Takara) as a vector, constructing a lentivirus or a transfection system of pLenti-HN3-Fc-ITGB1_TMD-ITGB1_ICD-PGK-Blasticidin S-WPRE, and stably infecting or transfecting into the ITGB1 knockout HEK293F cells.

The method of the present disclosure further comprises step (8) of loading scIL12 into the EVs to activate the immune system.

In the present disclosure, step (8) comprises constructing pIL12-Fc-TMD (NPTN-TMD)-ICD (EWI-F-ICD)-NanoLuc® plasmid for hscIL12 and mscIL12 using VB220306-1137jmq as a vector, co-transfecting the plasmid into the cells with Sleeping Beauty transposase expression vector pCMV-(CAT) T7-SB100X, co-infecting/transfecting into the ITGB1 knockout HEK293F cells with HN3 lentivirus and IL12 sleeping beauty plasmid, further screening and centrifuging cells to collect supernatant, purifying ITGB1⁻scIL12⁺HN3⁺ EV from the supernatant, and further performing deglycosylation to obtain ITGB1⁻scIL12⁺HN3⁺Deg EV.

In the present disclosure, step (8) comprises using HepG2 cells with high expression of human GPC3 protein for immunofluorescence verification, and using HepG2 cells, Hepa1-6, HEK293T, and modified Hepa1-6-hGPC3 and HEK293T-hGPC3 cells with high expression of human GPC3 protein for cell binding verification, to verify the targeting effect of EVs.

In some embodiments, the method further comprises treating human PBMCs with CD28 antibody, CD3 antibody and various concentrations of control EVs and measuring the expression of IFNγ stimulated by ITGB1⁻hscIL12⁺EV in human PBMCs in vitro by ELISA.

In some embodiments, the method further comprises treating mouse splenocytes with CD3 antibody and various concentrations of control EVs and measuring the expression of IFNγ stimulated by ITGB1⁻scIL12⁺HN3⁺EV in mouse splenocytes in vitro by ELISA.

In some embodiments, the method further comprises establishing an Hepa1-6-hGPC3 subcutaneous tumor model mouse and injecting ITGB1⁻mscIL12⁺HN3⁺Deg EV via the tail vein to verify the targeting effect of ITGB1⁻mscIL12⁺HN3⁺Deg EV (or referred to as HN3⁺EV) in the Hepa1-6-hGPC3 subcutaneous tumor model mouse.

In the present disclosure, the method further comprises inoculating tumor cells, performing administration on day 4 and day 7, measuring tumor volume and weight every two days, weighting tumors, and collecting tumors for pathological sectioning, to verify the tumor inhibitory effect of ITGB1⁻mscIL12⁺HN3⁺Deg EV in the Hepa1-6-hGPC3 subcutaneous tumor model mouse.

The present disclosure further provides an extracellular vesicle with reduced non-specific binding/endocytosis prepared by the method above.

In addition, the present disclosure further provides use of the extracellular vesicle prepared by the method above in the manufacture of a medicament.

In the present disclosure, the medicament is useful in the treatment of hepatocellular carcinoma with high expression of GPC3.

In addition, the present disclosure further provides a pharmaceutical composition, comprising the extracellular vesicle prepared by the method above as a raw material.

The present disclosure further provides a method for treating hepatocellular carcinoma, comprising administering the pharmaceutical composition of the present disclosure.

In the present disclosure, the hepatocellular carcinoma in the treatment method is hepatocellular carcinoma with high expression of GPC3, and the pharmaceutical composition is administered orally and/or by injection.

### Beneficial Effects:

Through the individual or combined knockout of ITGB1 and deglycosylation treatment, the present disclosure significantly reduces the non-specific binding/endocytosis of extracellular vesicles (EVs). This reduction minimizes the clearance associated with their use as drug delivery systems, thereby enhancing drug delivery efficiency and overall efficacy, allowing EVs to better target lesions.

By successfully and efficiently loading the cytokine IL-12 onto the extracellular vesicles, the present disclosure allows extracellular vesicles with low clearance to further possess the capability to activate the immune system, effectively achieving therapeutic goals. Additionally, further loading of an antibody allows for the targeting capability of these EVs. Thus, by combining the ITGB1 knockout, deglycosylation treatment, and loading of the cytokine IL-12 along with an antibody fragment, it is possible to achieve higher delivery efficiency. The targeting antibody fragment directs the IL12 EV to tumor sites to activate the local immune response. The expression of IL-12 on the surface of EV membranes can create an aggregating effect compared to single molecules of IL-12, significantly enhancing the stimulating function of IL-12. This allows the functionality to be achieved at lower doses, providing a certain degree of tumor inhibition, particularly against high GPC3-expressing hepatocellular carcinoma.

### BRIEF DESCRIPTION OF DRAWINGS

FIG. 1 shows the results of verification of ITGB1 knockout by flow cytometry in the present disclosure.

FIG. 2 shows the Western Blot identification of ITGB1 and CD81 expression of EVs in the present disclosure.

FIG. 3 shows the standard curve for EVs in the present disclosure.

FIG. 4 shows the verification of NanoLuc® loading position in the present disclosure.

FIG. 5 shows the binding/endocytosis of WT EVs or ITGB1 EVs with various recipient cells in the present disclosure, where a shows the binding/endocytosis of EVs with various recipient cells for 1 h, and b shows the binding/endocytosis of EVs with various recipient cells for 4 h.

In FIG. 6, a shows the plasma pharmacokinetic profile of EVs in the present disclosure, and b shows the drug concentration-time profile of EVs distributed in tissues in the present disclosure.

FIG. 7 shows the validation of EV deglycosylation by Western Blot in the present disclosure.

FIG. 8 shows the binding/endocytosis of deglycosylated ITGB1′EVs with various recipient cells in the present disclosure, where a shows the binding/endocytosis of EVs with various recipient cells for 1 h, and b shows the binding/endocytosis of EVs with various recipient cells for 4 h.

In FIG. 9, a shows the plasma pharmacokinetic profile of EVs in the present disclosure, and b shows the drug concentration-time profile of EVs distributed in tissues.

In FIG. 10, a shows the immunofluorescence detection of ITGB1⁻mscIL12⁺HN3⁺ EV binding with HepG2 cells in the present disclosure, and b shows the binding of ITGB1⁻mscIL12⁺HN3⁺ EVs with different cells in the present disclosure.

FIG. 11 shows the results of IFNγ secretion from human PBMCs stimulated by hscIL12⁺EV in the present disclosure.

FIG. 12 shows the results of IFNγ secretion from mouse splenocytes stimulated by mscIL12⁺EV in the present disclosure.

FIG. 13 shows the targeting capability of ITGB1⁻mscIL12⁺HN3⁺Deg EV in tumor-bearing mice in the present disclosure, where a shows the EV particle concentration per gram of tumor tissue, b shows the ratio of EV particle concentration per gram of tumor tissue to plasma EV particle concentration, and c shows the tumor size during the experiment.

FIG. 14 shows the tumor growth curve in the present disclosure.

FIG. 15 shows the HE staining of tumors in the present disclosure.

### DETAILED DESCRIPTION

Technical solutions of embodiments of the present disclosure are clearly and completely described below. Apparently, the embodiments described in the following are only some embodiments of the present disclosure, rather than all the embodiments. Any other embodiments obtained by those skilled in the art based on the embodiments in the present disclosure without any creative work fall within the scope of protection of the present disclosure.

### Example 1: Construction and Collection of EVs Derived from the ITGB1 Knockout HEK293F Suspension Cell Line, and In Vitro and In Vivo Functional Verification

### 1. Methods:

(1) ITGB1 knockout and cell line sorting: HEK293F suspension cells were transiently transfected with ITGB1 CRISP/Cas9/gRNA plasmid by using PEI transfection reagent. After 72 h, the cells were incubated with APC anti-human ITGB1 antibody, and subjected to flow cytometric sorting to obtain the APC negative cell population. The sorted cell population was then expanded, resulting in the ITGB1 knockout HEK293F cell line (ITGB1⁻HEK293F).
(2) Verification of ITGB1 knockout cell line: 2e5 HEK293F cells and 2e5 ITGB1⁻HEK293F cells were taken. 1E+5 HEK293F cells and 1E+5 TGB1⁻HEK293F cells were mixed as Mix. After centrifugation, the cells were resuspended in 100 μl of PBS. Then, 1 μl of APC anti-human ITGB1 antibody was added, and the mixture was incubated in the dark at 20 °C.±5 °C for 30 min. The cells were washed three times with phosphate buffered saline (PBS), resuspended in 300 μl of PBS, and analyzed using a flow cytometer. Additionally, after purifying the EVs, Western Blot analysis was conducted to identify changes in ITGB1 expression on the ITGB1⁻EVs.
(3) NanoLuc® loading: For NanoLuc®, the pFc-TMD (NPTN-TMD)-ICD (EWI-F-ICD)-NanoLuc® plasmid was constructed using VB220306-1137jmq (Yunzhou Biotech, Sleeping Beauty Expression Vector) as a vector. This plasmid was co-transfected with the Sleeping Beauty (SB) transposase expression vector pCMV-(CAT) T7-SB100X into the cells. After 72 h, Zeocin™ antibiotic at a final concentration of 100 μg/ml was added until all cells in a non-stably transfected group died (approximately three days). The cells were then expanded, and the supernatant was collected by centrifugation at 5000 rpm for 30 min, followed by purification of the EVs from the supernatant. A standard curve for EV particle concentration-NanoLuc® enzymatic activity was established to verify the NanoLuc® activity of the EVs. Triton™ X-100 and Proteinase K were used to treat the EVs to confirm the loading location of NanoLuc® within the EVs.

The Fc amino acid sequence involved (SEQ ID NO. 1):
AESKYGPPCPPCPAPEAAGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLG
NPTN-TMD sequence involved:
LWPFLGILAEIIILVVIIVVY
EWI-F-ICD sequence involved:
SSHWCCKKEVQETRRERRRLMSMEMD
The NanoLuc ® amino acid sequence involved (SEQ ID NO. 2):
MVFTLEDFVGDWRQTAGYNLDQVLEQGGVSSLFQNLGVSVTPIQRIVLSGENGLKIDIH
VIIPYEGLSGDQMGQIEKIFKVVYPVDDHHFKVILHYGTLVIDGVTPNMIDYFGRPYEGIA
VFDGKKITVTGTLWNGNKIIDERLINPDGSLLFRVTINGVTGWRLCER

(4) Verification of changes in binding/endocytosis of ITGB1 knockout HEK293F cell-derived EVs with different cells at cellular level: Recipient cells were inoculated into a 24-well plate at 0.5E+5 per well. The following day, the EV concentration was adjusted to 1E+10 p/ml and then diluted in complete culture medium to a concentration of 1E+9 p/ml. After removing the supernatant from the cells, 0.5 ml of EVs was added, and the cells were incubated at 37 °C for either 1 h or 4 h. At the end of the incubation period, the supernatant was gently removed, and the cells were washed once with 1 ml of PBS. Then, 1 ml of EDTA was added, and the cells were incubated for 10 min to digest. The cells were collected and centrifuged at 1000 rpm for 5 min. After washing once with 1 ml of 2% FBS/PBS, the cells were resuspended in 200 μl of 2% FBS/PBS. 50 μl was transferred to a white 96-well plate, and 50 μl of NanoLuc® detection substrate was added to measure the NanoLuc® activity in the samples.
(5) In vivo verification: To evaluate the effect of ITGB1 knockout on the tissue distribution of EVs in major organs in vivo, EVs were injected via the tail vein at a dose of 5E+10 particles per mouse. Plasma was collected from peripheral blood at 5, 15, 30, 60, 120, and 240 minutes, and liver, spleen, lung, and kidney tissues were homogenized. NanoLuc® enzymatic activity was detected in both plasma and tissue homogenates. A standard curve for EV particle concentration-NanoLuc® enzymatic activity was established to calculate the EV particle concentration in the tissues.

### 2. Results:

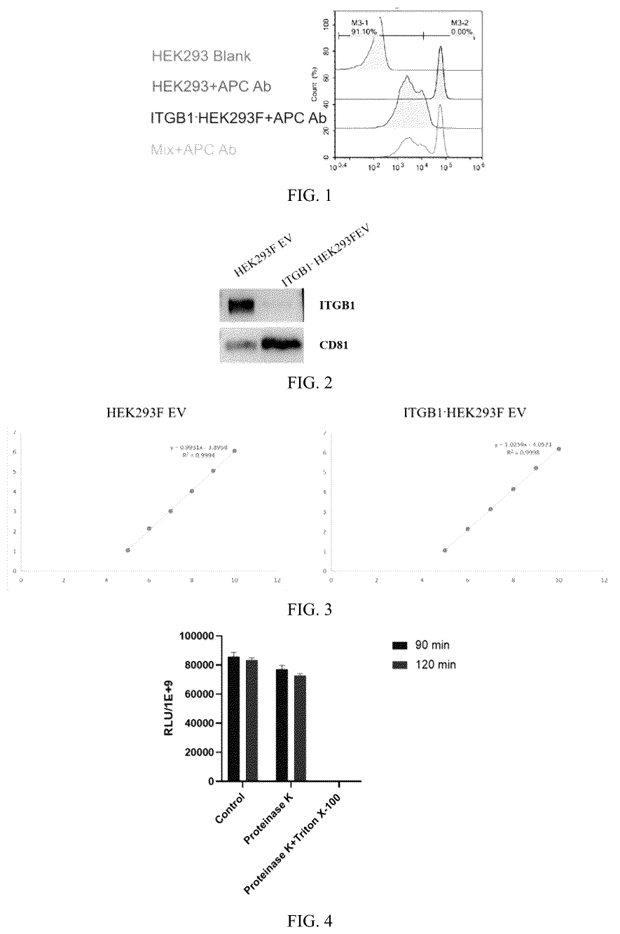

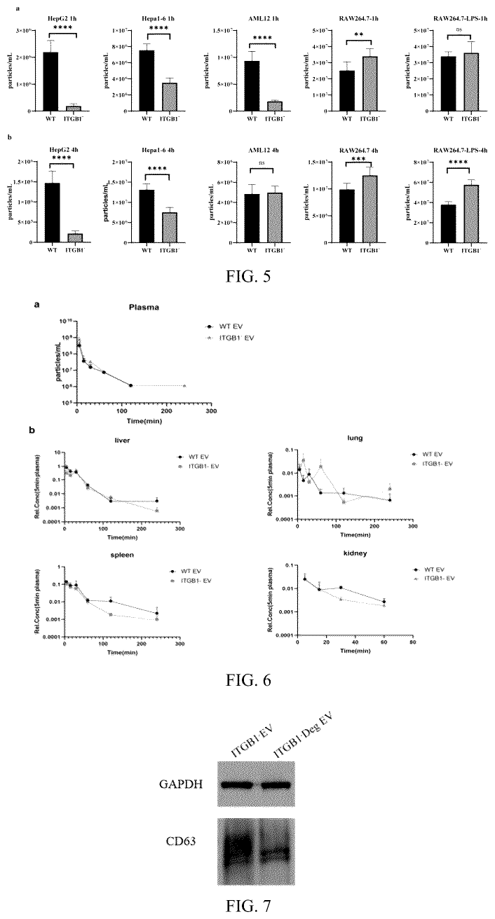

As shown in FIGS. 1-6 and Tables 1-4, the wild-type HEK293F cells highly expressed ITGB1. After ITGB1 knockout, flow cytometry analysis indicated a significant decrease in APC fluorescence after the cells were incubated with APC anti-human ITGB1 antibody. The mixed sample of wild-type and ITGB1 knockout cells at a 1:1 ratio clearly separated into an APC strong-positive cell population and the ITGB1 knockout cell population, indicating successful ITGB1 knockout. Western Blot analysis demonstrated that both WT EV and ITGB1 EV samples expressed the EV marker CD81. WT EV expressed ITGB1, whereas ITGB1 EV did not express the ITGB1 protein, confirming successful ITGB1 knockout at the EV level.

The enzymatic activity of WT EV was approximately 1E+05 per 10⁹ EV particles, while ITGB1 EV exhibited enzymatic activity of approximately 1.6 E+05 per 10⁹ EV particles. A linear relationship between enzymatic activity and EV particle concentration was observed when the particle concentration ranged from 1E+5 to 1E+10.

Proteinase K treatment alone digested the free NanoLuc® or NanoLuc® located outside the EV membrane. After the addition of Triton™ X-100, the EV membrane structure was disrupted, allowing Proteinase K to enter the EV and digest the internal NanoLuc®. Experimental results showed that the enzymatic activity of EVs treated solely with Proteinase K only slightly decreased over time, while the enzymatic activity of EVs treated with both Triton™ X-100 and Proteinase K was nearly undetectable after 90 min, indicating that NanoLuc® was loaded within the EVs, consistent with the loading target.

Following ITGB1 knockout, the binding/endocytosis of EVs by human liver-derived HepG2 cells or mouse liver-derived Hepa1-6 and AML12 cells decreased at 1 h. However, the binding of EVs by mouse monocyte RAW264.7 cells, regardless of LPS activation, remained the same or increased. At 4 h, the binding/endocytosis of ITGB1 EV by HepG2 and Hepa1-6 cells remained lower than that of wild-type HEK293 EV, while the binding/endocytosis for both types of EVs by AML12 cells was similar. The 4-hour results for RAW264.7 cells were similar to the 1-hour results, indicating that ITGB1 knockout promoted the binding/endocytosis of EVs. This suggested that ITGB1 knockout had varying effects on binding/endocytosis for different recipient cells in vitro, while it could reduce the binding/endocytosis of EVs by liver-derived cells to a certain extent.

In vivo verification: Results indicated that ITGB1 knockout did not significantly alter the distribution of EVs in the major organs (liver, spleen, lung, kidney) and blood of mice.

TABLE 1
NanoLuc ® loading position verification

| | 90 min | | | 120 min | | |
|---|---|---|---|---|---|---|
| Control | 82453 | 88070 | 87025 | 82000 | 85121 | 83367 |
| Proteinase K | 74110 | 78642 | 78881 | 71391 | 73899 | 73352 |
| Proteinase K+ Triton ™ X-100 | 84 | 83 | 87 | 38 | 41 | 60 |

TABLE 2
Binding/endocytosis of WT EV and ITGB1⁻EV with various recipient cells after 1 h or
4 h incubation

| HepG2 | | Hepa1-6 | | AML12 1 h | | RAW264.7 | | RAW264.7-lps | |
|---|---|---|---|---|---|---|---|---|---|
| WT | ITGB1⁻ | WT | ITGB1⁻ | WT | ITGB1⁻ | WT | ITGB1⁻ | WT | ITGB1⁻ |
| 1810000 | 142000 | 6730000 | 2960000 | 5670000 | 1380000 | 2.27E+07 | 3.03E+07 | 3.25E+07 | 3.67E+07 |
| 2230000 | 124000 | 8890000 | 4250000 | 9680000 | 1940000 | 2.48E+07 | 2.65E+07 | 3.74E+07 | 3.06E+07 |
| | 118000 | 7480000 | 4090000 | 1.03E+07 | 1520000 | 2.41E+07 | 3.26E+07 | 3.09E+07 | 3.16E+07 |
| 2650000 | 178000 | 7240000 | 3900000 | 1.01E+07 | 1910000 | 2.04E+07 | 3.42E+07 | 3.18E+07 | 3.45E+07 |
| 1510000 | 154000 | 8350000 | 3570000 | 1.18E+07 | 1870000 | 2.04E+07 | 2.97E+07 | 3.18E+07 | 3.82E+07 |
| 2720000 | 384000 | 7540000 | 3720000 | 8530000 | 2150000 | 2.62E+07 | 3.20E+07 | 3.35E+07 | 2.91E+07 |
| 2180000 | 196000 | 7680000 | 3120000 | 8980000 | 1850000 | 1.89E+07 | 3.75E+07 | 3.08E+07 | 2.90E+07 |
| 2260000 | 208000 | 6470000 | 2510000 | 9680000 | 1970000 | 2.69E+07 | 3.84E+07 | 3.81E+07 | 4.03E+07 |
| | | | | | | 2.97E+07 | 4.20E+07 | 3.61E+07 | 3.88E+07 |
| | | | | | | 3.76E+07 | 3.71E+07 | 3.70E+07 | 5.21E+07 |
| HepG2 4 h | | Hepa1-6 4 h | | AML12 4 h | | RAW264.7 4 h | | RAW264.7-lps 4 h | |
| WT | ITGB1⁻ | WT | ITGB1⁻ | WT | ITGB1⁻ | WT | ITGB1⁻ | WT | ITGB1⁻ |
| 1030000 | 268000 | 1.28E+07 | 6730000 | 3960000 | 3530000 | 1.09E+07 | 1.38E+07 | 3.59E+07 | 6.25E+07 |
| 1480000 | 130000 | 1.45E+07 | 8320000 | 4320000 | 4820000 | 1.17E+07 | 1.31E+07 | 3.83E+07 | 6.25E+07 |
| 1160000 | 166000 | 1.31E+07 | 7980000 | 3430000 | 5460000 | 1.12E+07 | 1.52E+07 | 3.85E+07 | 6.29E+07 |
| 1340000 | 136000 | 1.51E+07 | 9430000 | 4650000 | 5030000 | 9250000 | 1.27E+07 | 3.84E+07 | 5.84E+07 |
| 1580000 | 202000 | 1.35E+07 | 8060000 | 5100000 | 4920000 | 1.01E+07 | 1.30E+07 | 3.33E+07 | 6.02E+07 |
| 1650000 | 238000 | 1.34E+07 | 5210000 | 6260000 | 5380000 | 7820000 | 1.25E+07 | 3.76E+07 | 4.90E+07 |
| 1570000 | 305000 | 1.24E+07 | 7370000 | 5490000 | 5060000 | 1.04E+07 | 1.05E+07 | 3.94E+07 | 5.62E+07 |
| 1970000 | 286000 | 1.04E+07 | 7230000 | 5610000 | 5610000 | 9160000 | 1.05E+07 | 3.34E+07 | 5.94E+07 |
| | | | | | | 9160000 | 1.10E+07 | 4.24E+07 | 5.38E+07 |
| | | | | | | 9230000 | 1.29E+07 | 4.23E+07 | 5.21E+07 |

TABLE 3
Plasma pharmacokinetic profile of EVs

| | WT EV | ITGB1⁻ EV |
|---|---|---|
| 5 | 3.25E+08 | 7.65E+08 |
| 15 | 3.65E+07 | 4.36E+07 |
| 30 | 1.53E+07 | 3.45E+07 |
| 60 | 7.47E+06 | 8.08E+06 |
| 120 | 1.16E+06 | 1.24E+06 |
| 240 | BQL | 1.19E+06 |

TABLE 4
Drug concentration-time profile of EV tissue distribution

| | Liver | | Spleen | | Lung | | Kidney | |
|---|---|---|---|---|---|---|---|---|
| | WT EV | ITGB1⁻EV | WT EV | ITGB1⁻EV | WT EV | ITGB1⁻EV | WT EV | ITGB1⁻EV |
| 5 | 0.830 | 0.325 | 0.142 | 0.105 | 0.0140 | 0.0154 | 0.0251 | 0.0252 |
| 15 | 0.437 | 0.228 | 0.086 | 0.070 | 0.0045 | 0.0356 | 0.0090 | 0.0093 |
| 30 | 0.380 | 0.413 | 0.090 | 0.057 | 0.0090 | 0.0039 | 0.0109 | 0.0035 |
| 60 | 0.041 | 0.026 | 0.012 | 0.010 | 0.0013 | 0.0190 | 0.0027 | 0.0019 |
| 120 | 0.003 | 0.005 | 0.011 | 0.002 | 0.0013 | 0.0005 | BQL | BQL |
| 240 | 0.003 | 0.001 | 0.002 | 0.001 | 0.0007 | 0.0020 | BQL | BQL |

### Example 2: Deglycosylated HEK293F EVs Exhibited Significantly Improved In Vitro and In Vivo Pharmacokinetic Properties

The majority of proteins on the EV surface are glycosylated, such as CD63, one of the classic EV markers. Glycoproteins are involved in intracellular and extracellular signal transduction, intercellular interactions, and endocytosis through the regulation and control of biological processes such as cell adhesion, cellular ligand binding, and receptor dimerization. In other words, glycoproteins are recognized as one of the major important pathways for EV binding/endocytosis with recipient cells. N-glycosylation is one of the primary modifications of protein glycosylation. Therefore, in order to reduce the nonspecific binding of EVs, N-glycosylation on EV surfaces was removed using the PNGase F enzyme. The changes in binding/endocytosis of deglycosylated EVs at cellular level in vitro, as well as the changes in pharmacokinetics and tissue distribution in vivo were verified.

### 1. Methods:

(1) Deglycosylation of EVs (DegEV) and verification: 10 μl of PNGase F and 100 μl of 10×Reaction Buffer were added per 2E+10 EVs. The system was supplemented to 1 ml with PBS, with the EV concentration of 2E+10 p/ml, and incubated at 37 °C for 1 h. CD63 and GAPDH were detected by Western Blot.
(2) Verification of changes in binding/endocytosis of deglycosylated EVs with different recipient cells at cellular level: Cells were inoculated into a 24-well plate at 0.5E+5 per well. The following day, the EV concentration was adjusted to 1E+10 p/ml with PBS and then diluted in DMEM medium containing 10% to a concentration of 1E9 p/ml. After removing the supernatant from the cells, 0.5 ml of DMEM complete medium with EVs was added, and the cells were incubated at 37 °C for either 1 h or 4 h. At the end of the incubation period, the supernatant was gently removed, and the cells were washed once with 1 ml of PBS. Then, 1 ml of EDTA was added, and the cells were incubated for 10 min to digest. The cells were collected and centrifuged at 1000 rpm for 5 min. After washing once with 1 ml of 2% FBS/PBS, the cells were resuspended in 200 μl of 2% FBS/PBS. 50 μl was transferred to a white 96-well plate, and 50 μl of NanoLuc® detection substrate was added to measure the NanoLuc® activity in the samples.
(3) In vivo verification: To evaluate the effect of deglycosylation on the tissue distribution of EVs in major organs in vivo, EVs were injected via the tail vein at a dose of 5E+10 particles per mouse. Plasma was collected from peripheral blood at 5, 15, 30, 60, 120, and 240 minutes, and the liver, spleen, lung, and kidney tissues were homogenized. NanoLuc® enzymatic activity was detected in both plasma and tissue homogenates. A standard curve for EV particle concentration-NanoLuc® enzymatic activity was established to calculate the EV particle concentration in the tissues.

### 2. Results

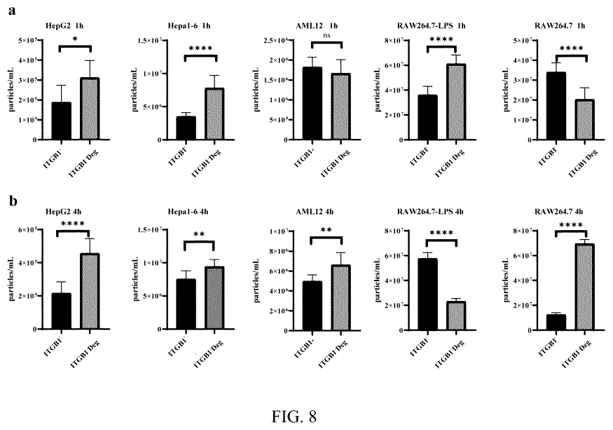

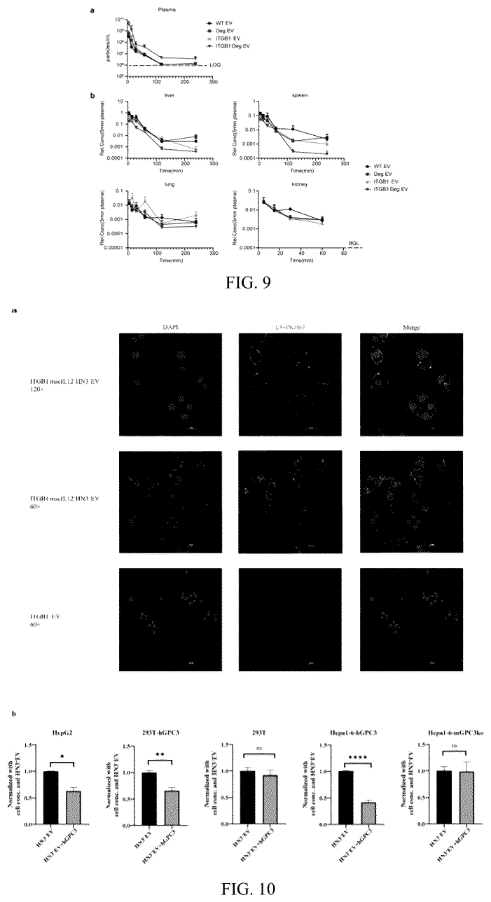

As shown in FIGS. 7-9 and Tables 5-7, deglycosylated EVs were analyzed by Western Blot for CD63 and GAPDH. The results showed that the GAPDH detection was consistent across both types of EVs. CD63 displayed diffuse bands due to varying degrees of glycosylation; however, after deglycosylation, the bands became relatively concentrated, confirming successful deglycosylation.

In vitro verification at cellular level: The experimental results of binding/endocytosis of ITGB1 knockout combined with deglycosylated EVs (ITGB1 DegEV) and ITGB1 knockout EVs (ITGB1 EV) with different recipient cells showed that deglycosylation promoted the binding of EVs to HepG2 and Hepa1-6 cells. In contrast, for AML12 and RAW264.7 cells, the binding/endocytosis of ITGB1 DegEV was less than that of ITGB1 EV at 1 h; however, the binding/endocytosis of DegEV was greater at 4 h. In LPS-stimulated RAW264.7 cells, the binding/endocytosis of DegEV was greater than that of ITGB1 EV at 1 h, but it was lower than that of ITGB1⁻EV at 4 h. This indicated that the effect of deglycosylation on the binding/endocytosis capability of EVs with different recipient cells was inconsistent.

In vivo verification: The in vivo results demonstrated that neither deglycosylation (Deg EV) nor ITGB1 knockout (ITGB1 EV) alone significantly altered the tissue distribution or plasma circulation time of EVs. However, the combination of deglycosylation and ITGB1 knockout significantly increased the circulation time of EVs in plasma while correspondingly reducing their accumulation in major organs (liver, spleen, lung, and kidney).

TABLE 5
Binding/endocytosis of deglycosylated ITGB1⁻EV with different recipient cells

| HepG2 1 h | | Hepa1-6 1 h | | AML12 1 h | | RAW264.7 1 h | | RAW264.7-LPS 1 h | |
|---|---|---|---|---|---|---|---|---|---|
| | ITGB1⁻ | | ITGB1⁻ | | ITGB1⁻ | | ITGB1⁻ | | ITGB1⁻ |
| ITGB1⁻ | Deg | ITGB1⁻ | Deg | ITGB1⁻ | Deg | ITGB1⁻ | Deg | ITGB1⁻ | Deg |
| 142000 | 319000 | 2960000 | 5160000 | 1380000 | 1620000 | 3.03E+07 | 3.12E+07 | 3.67E+07 | 7.32E+07 |
| 124000 | 202000 | 4250000 | 7180000 | 1940000 | 1100000 | 2.65E+07 | 1.92E+07 | 3.06E+07 | 5.67E+07 |
| 118000 | 244000 | 4090000 | 1.08E+07 | 1520000 | 1500000 | 3.26E+07 | 2.81E+07 | 3.16E+07 | 6.40E+07 |
| 178000 | 235000 | 3900000 | 7040000 | 1910000 | 1400000 | 3.42E+07 | 2.33E+07 | 3.45E+07 | 5.99E+07 |
| 154000 | 427000 | 3570000 | 7590000 | 1870000 | 1920000 | 2.97E+07 | 2.06E+07 | 3.82E+07 | 5.69E+07 |
| 384000 | 394000 | 3720000 | | 2150000 | 2100000 | 3.20E+07 | 1.79E+07 | 2.91E+07 | 6.36E+07 |
| 196000 | 402000 | 3120000 | | 1850000 | 2060000 | 3.75E+07 | 1.51E+07 | 2.90E+07 | 6.01E+07 |
| 208000 | 269000 | 2510000 | 9070000 | 1970000 | 1650000 | 3.84E+07 | 1.43E+07 | 4.03E+07 | 6.79E+07 |
| | | | | | | 4.20E+07 | 1.32E+07 | 3.88E+07 | 6.22E+07 |
| | | | | | | 3.71E+07 | 2.02E+07 | 5.21E+07 | 4.73E+07 |
| | | Hepa1-6 4 h | | AML12 4 h | | RAW264.7 4 h | | RAW264.7-LPS 4 h | |
| HepG2 4 h | | | ITGB1⁻ | | ITGB1⁻ | | ITGB1⁻ | | ITGB1⁻ |
| ITGB1⁻ | ITGB1⁻Deg | ITGB1⁻ | Deg | ITGB1⁻ | Deg | ITGB1⁻ | Deg | ITGB1⁻ | Deg |
| 268000 | 494000 | 6730000 | 7610000 | 3530000 | 5920000 | 1.38E+07 | 6.92E+07 | 6.25E+07 | 1.97E+07 |
| 130000 | 436000 | 8320000 | 1.07E+07 | 4820000 | 5260000 | 1.31E+07 | 6.96E+07 | 6.25E+07 | 2.20E+07 |
| 166000 | 386000 | 7980000 | 8870000 | 5460000 | 6470000 | 1.52E+07 | 7.21E+07 | 6.29E+07 | 2.11E+07 |
| 136000 | 361000 | 9430000 | 9470000 | 5030000 | 5280000 | 1.27E+07 | 6.82E+07 | 5.84E+07 | 2.72E+07 |
| 202000 | 561000 | 8060000 | 1.00E+07 | 4920000 | 8210000 | 1.30E+07 | 7.24E+07 | 6.02E+07 | 2.06E+07 |
| 238000 | 602000 | 5210000 | 1.05E+07 | 5380000 | 5910000 | 1.25E+07 | 6.69E+07 | 4.90E+07 | 2.45E+07 |
| 305000 | 444000 | 7370000 | 8350000 | 5060000 | 8040000 | 1.05E+07 | 6.83E+07 | 5.62E+07 | 2.45E+07 |
| 286000 | 369000 | 7230000 | 9970000 | 5610000 | 7850000 | 1.05E+07 | 6.30E+07 | 5.94E+07 | 2.48E+07 |
| | | | | | | 1.10E+07 | 7.18E+07 | 5.38E+07 | 2.41E+07 |
| | | | | | | 1.29E+07 | 7.44E+07 | 5.21E+07 | 2.33E+07 |

TABLE 6
Plasma pharmacokinetic profile of EVs

| | WT EV | Deg EV | ITGB1⁻ EV | ITGB1⁻Deg EV |
|---|---|---|---|---|
| 5 | 3.25E+08 | 6.27E+08 | 7.65E+08 | 3.87E+09 |
| 15 | 3.65E+07 | 1.47E+08 | 4.36E+07 | 1.26E+09 |
| 30 | 1.53E+07 | 2.27E+07 | 3.45E+07 | 6.48E+07 |
| 60 | 7.47E+06 | 9.09E+06 | 8.08E+06 | 4.08E+07 |
| 120 | 1.16E+06 | 1.18E+06 | 1.24E+06 | 4.54E+06 |
| 240 | BQL | 1.38E+06 | 1.19E+06 | 3.78E+06 |

TABLE 7
Drug concentration-time profile of EV tissue distribution

| | Liver | | | | Spleen | | | |
|---|---|---|---|---|---|---|---|---|
| min | WT EV | Deg EV | ITGB1⁻EV | ITGB1⁻Deg EV | WT EV | Deg EV | ITGB1⁻EV | ITGB1⁻Deg EV |
| 5 | 0.8297 | 0.7873 | 0.3253 | 0.1708 | 0.1421 | 0.1374 | 0.1053 | 0.0490 |
| 15 | 0.4369 | 0.3484 | 0.2275 | 0.1647 | 0.0858 | 0.0662 | 0.0697 | 0.0438 |
| 30 | 0.3804 | 0.1887 | 0.4132 | 0.0485 | 0.0904 | 0.0434 | 0.0570 | 0.0187 |
| 60 | 0.0415 | 0.0348 | 0.0257 | 0.0202 | 0.0122 | 0.0077 | 0.0100 | 0.0112 |
| 120 | 0.0029 | 0.0031 | 0.0052 | 0.0007 | 0.0111 | 0.0016 | 0.0018 | 0.0003 |
| 240 | 0.0030 | 0.0082 | 0.0006 | 0.0004 | 0.0022 | 0.0029 | 0.0010 | 0.0002 |
| | Lung | | | | Kidney | | | |
| | | Deg | | | | Deg | | |
| min | WT EV | EV | ITGB1⁻ EV | ITGB1⁻Deg EV | WT EV | EV | ITGB1⁻ EV | ITGB1⁻Deg EV |
| 5 | 0.0140 | 0.0126 | 0.0154 | 0.0138 | 0.0251 | 0.0251 | 0.0252 | 0.0260 |
| 15 | 0.0045 | 0.0045 | 0.0356 | 0.0078 | 0.0090 | 0.0096 | 0.0093 | 0.0123 |
| 30 | 0.0090 | 0.0047 | 0.0039 | 0.0039 | 0.0109 | 0.0041 | 0.0035 | 0.0034 |
| 60 | 0.0013 | 0.0017 | 0.0190 | 0.0032 | 0.0027 | 0.0030 | 0.0019 | 0.0030 |
| 120 | 0.0013 | 0.0004 | 0.0005 | 0.0003 | BQL | BQL | BQL | BQL |
| 240 | 0.0007 | 0.0006 | 0.0020 | 0.0003 | BQL | BQL | BQL | BQL |

### Example 3: Construction of ITGB1⁻scIL12⁺HN3⁺Deg EV and Verification of their In Vivo and In Vitro Functions

By loading an antibody into EVs, they gain specific targeting capabilities, and by further loading scIL12, they can activate the immune system. EVs loaded with both an antibody and scIL12 can achieve a certain level of tumor suppression in vivo.

### 1. Methods

(1) Construction of hscIL12 and mscIL12 Sleeping Beauty expression vectors and HN3 lentivirus: For hscIL12 and mscIL12, pIL12-Fc-TMD (NPTN-TMD)-ICD (EWI-F-ICD)-NanoLuc® plasmid was constructed using VB220306-1137jmq (Yunzhou Biotech, Sleeping Beauty Expression Vector) as the vector. This plasmid was co-transfected into cells with the Sleeping Beauty (SB) transposase expression vector pCMV-(CAT) T7-SB100X. For HN3, the lentivirus pLenti-HN3-Fc-ITGB1_TMD-ITGB1_ICD-PGK-Blasticidin S-WPRE was constructed. The HN3 lentivirus and IL12 Sleeping Beauty plasmid were either separately or co-infected/transfected into ITGB1 knockout HEK293F cells. Following transfection, the cells were cultured for 72 h, after which puromycin and/or Blasticidin S antibiotics were added for selection over another 72 h. The supernatant was collected by centrifugation at 5000 rpm for 30 min, and EVs were purified from the supernatant. The resulting EVs were designated as ITGB1⁻hscIL12⁺EV (or ITGB1⁻mscIL12⁺EV) and ITGB1⁻hscIL12⁺HN3⁺EV (or ITGB1⁻mscIL12⁺HN3⁺EV). After further deglycosylation, ITGB1⁻hscIL12⁺HN3⁺Deg EV (or ITGB1⁻mscIL12⁺HN3⁺Deg EV) was obtained. The aforementioned EVs were referred to as hscIL12⁺EV in in vitro experiments with human PBMCs, as HN3⁺EV in in vitro binding assays with hGPC3+ cells, and as mscIL12⁺EV in murine splenic lymphocyte stimulation assays.

mscIL12 sequence involved:
mscIL12-P35 (SEQ ID NO. 3):
MCQSRYLLFL ATLALLNHLS
LARVIPVSGPARCLSQSRNLKTTDDMVKTAREKLKHYSC TAEDIDHEDI TRDQTSTLKT
CLPLELHKNE SCLATRETSSTTRGSCLPPQ KTSLMMTLCL
GSIYEDLKMYQTEFQAINAALQNHNHQQIILDKGMLVAIDELMQSLNHNGETLRQKPPV
G EADPYRVKMK LCILLHAFSTRVVTINRVMG YLSSA
mscIL12-P40 (SEQ ID NO. 4):
MCPQKLTISWFAIVLLVSPLMAMWELEKDVYVVEVDWTPDAPGETVNLTCDTPEEDDI
TWTSDQRHGVIGSGKTLTITVKEFLDAGQYTCHKGGETLSHSHLLLHKKENGIWSTEILK
NFKNKTFLKCEAPNYSGRFTCSWLVQRNMDLKFNIKSSSSSPDSRAVTCGMASLSAEKV
TLDQRDYEKYSVSCQEDVTCPTAEETLPIELAL
EARQQNKYENYSTSFFIRDIIKPDPPKNLQ
MKPLKNSQVEVSWEYPDSWSTPHSYFSLKFFVRIQRKKEKMKETEEGCNQKGAFLVEK
TSTEVQCKGGNV CVQAQDRYYN SSCSKWACVP CRVRS
hscIL12 sequence involved:
hscIL12-P35 (SEQ ID NO. 5):
RNLPVATPDPGMFPCLHHSQNLLRAVSNMLQKARQTLEFYPCTSEEIDHEDITK
DKTSTVEACLPLELTKNESCLNSRETSFITNGSCLASRKTSFMMALCLSSIYEDLKMYQV
EFKTMNAKLLMDPKRQIFLDQNMLA VIDELMQALNFNSETVPQKSSLEEPDFYKTKIKL
CILLHAFRIRAVTIDRVMSYLNAS
hscIL12-P40 (SEQ ID NO. 6):
IWELKKDVYVVELDWYPDAPGEMVVLTCDTPEEDGITWTLDQSSEVLGSGKT
LTIQVKEFGDAGQYTCHKGGEVLSHSLLLLHKKEDGIWSTDILKDQKEPKNKTFLRCEA
KNYSGRFTCWWLTTISTDLTFSVKSSRGSSDPQGVTCGAATLSAERVRGDNKEYEYSVE
CQEDSACPAAEESLPIEVMVDAVHKLKYENYTSSFFIRDIIKPDPPKNLQLKPLKNSRQVE
VSWEYPDTWSTPHSYFSLTFCVQVQGKSKREKKDRVFTDKTSATVICRKNASISVRAQD
RYYSSSWSEWASVPCS
The ITGB1 amino acid sequence involved (SEQ ID NO. 7):
QTDENRCLKANAKSCGECIQAGPNCGWCTNSTFLQEGMPTSARCDDLEALKKKGCPPD
DIENPRGSKDIKKNKNVTNRSKGTAEKLKPEDITQIQPQQLVLRLRSGEPQTFTLKFKRA
EDYPIDLYYLMDLSYSMKDDLENVKSLGTDLMNEMRRITSDFRIGFGSFVEKTVMPYIS
TTPAKLRNPCTSEQNCTSPFSYKNVLSLTNKGEVFNELVGKQRISGNLDSPEGGFDAIMQ
VAVCGSLIGWRNVTRLLVESTDAGFHFAGDGKLGGIVLPNDGQCHLENNMYTMSHYY
DYPSIAHLVQKLSENNIQTIFAVTEEFQPVYKELKNLIPKSAVGTLSANSSNVIQLIIDAYN
SLSSEVILENGKLSEGVTISYKSYCKNGVNGTGENGRKCSNISIGDEVQFEISITSNKCPKK
DSDSFKIRPLGFTEEVEVILQYICECECQSEGIPESPKCHEGNGTFECGACRCNEGRVGRH
CECSTDEVNSEDMDAYCRKENSSEICSNNGECVCGQCVCRKRDNTNEIYSGKFCECDNF
NCDRSNGLICGGNGVCKCRVCECNPNYTGSACDCSLDTSTCEASNGQICNGRGICECGV
CKCTDPKFQGQTCEMCQTCLGVCAEHKECVQCRAFNKGEKKDTCTQECSYFNITKVES
RDKLPQPVQPDPVSHCKEKDVDDCWFYFTYSVNGNNEVMVHVVENPECPTGPD
HN3 sequence involved (SEQ ID NO. 8):
MQVQLVQSGGGLVQPGGSLRLSCAASYFDFDSYEMSWVRQAPGKGLEWIGSIYHSGST
YYNPSLKSRVTISRDNSKNTLYLQMNTLRAEDTATYYCARVNMDRFDYWGQGTLVTV
SSS

(2) Verification of targeting capability of ITGB1⁻mcIL12⁺HN3⁺ EV (or referred to as HN3⁺EV): HepG2 cells with high expression of human GPC3 protein were used to verify the targeting capability of EVs by immunofluorescence assay. HepG2 cells, Hepa1-6 cells, 293T cells, and modified Hepa1-6-hGPC3 and 293T-hGPC3 cells with high expression of human GPC3 protein were subjected to cell binding assay for verification.

Immunofluorescence assay: The cells were pre-seeded in a 24-well plate containing glass slides. The following day, the supernatant was removed, and the cells were washed once with PBS. They were fixed with paraformaldehyde for 10 min, followed by three washes with PBS, each for 5 min. A 1 ml solution of 5% BSA/PBS was added for blocking at room temperature for 2 h, and the cells were washed three times with PBS, each for 5 min. WT EVs were added at a ratio of cell: EV=1:30000 and incubated at 25 °C.±5 °C for 2 h. The cells were washed three times with PBS, each for 5 min. Either ITGB1⁻HEK293 F EV or ITGB1⁻mscIL12⁺HN3⁺EV, at a quantity of 5E+10 particles, was added to 1 ml of PKH67 dye solution with 6 μl of dye, and incubated at 25 °C.±5 °C in the dark for 30 min. Free dye was removed using core700. A quantity of 2E+10 particles was added to the slides in a volume of 1 ml, and incubated at 4 °C for 4 h. The cells were washed five times with PBST, each for 5 min. After staining with DAPI for 3 min, the cells were washed three times, covered with a cover slip, and photographed once the slides were dry.

(3) Cell binding: The cells were digested with EDTA and then centrifuged at 1000 rpm for 5 min. They were resuspended in culture medium at a concentration of 4E6 cells per 1.5 ml and mixed with 400 μl of WT EV, which was stored at a concentration of 3.88E11 p/ml. The mixture was gently mixed and rotated at 4 °C and incubated for 1 h. For the ITGB1⁻mscIL12⁺HN3⁺EV+hGPC3 protein group, pre-incubation was performed at 4 °C for 30 min. The ITGB1⁻mscIL12⁺HN3⁺EV was stored at a concentration of 2.15E+11; 367 μl was taken and diluted in 4000 μl of PBS to achieve a final concentration of 1.8E10 p/ml. For the ITGB1⁻mscIL12⁺HN3⁺EV+hGPC3 group, 1400 μl of EV was mixed with 100 μl of human GPC3 protein at a storage concentration of 100 μg/ml. For the ITGB1⁻mscIL12⁺HN3⁺ EV+Fc-hlgG1 group, 1400 μl of EV was mixed with 100 μl of human Fc-IgG1 protein at a storage concentration of 100 μg/ml. The cells that were pre-blocked with WT EV were aliquoted in 100 μl portions, with approximately 2E+5 cells per tube, and 150 μl of EV was added according to the group. They were incubated at 4 °C for 1 h. The cells were washed twice with 2% FBS/PBS, with 1 ml each time. After removing the supernatant, 200 μl of PBS was added for resuspension, and 30 μl of cells was mixed with 30 μl of NanoLuc® detection substrate for detection of NanoLuc® enzyme activity.

In vitro stimulation of human PBMCs with ITGB1⁻hscIL12⁺EV to express IFNγ: 200,000 human PBMCs were added to a 96-well plate, along with 1 μg/ml of anti-CD3 antibody, 1 μg/ml of anti-CD28 antibody, and varying concentrations of control EV (not loaded with hscIL12) or hscIL12⁺EV or hIL12 protein. After 4 days, the cell supernatant was collected and the concentration of human IFNγ in the supernatant was detected using an ELISA kit (Shanghai Enzyme-Link Biotechnology, EK-180-96).

(4) In vitro stimulation of mouse splenocytes with ITGB1⁻mscIL12⁺HN3⁺EV (or referred to as mscIL12⁺EV) to express IFNγ: 200,000 mouse splenocytes were added to a 96-well plate, along with 1 ng/ml of anti-CD3 antibody and varying concentrations of control EV (not loaded with mscIL12) or mscIL12⁺EV. After 4 days, the cell supernatant was collected and the concentration of mouse IFNγ in the supernatant was detected using an ELISA kit (Shanghai Enzyme-Link Biotechnology, EK-180-96).

(5) Targeting of ITGB1⁻mscIL12⁺HN3⁺Deg EV (or referred to as HN3⁺EV) in a Hepa1-6-hGPC3 subcutaneous tumor model mouse: The Hepa1-6-hGPC3 subcutaneous tumor model was established by inoculating 5×10⁶ cells per 100 μl into the axillary subcutaneous tissue of C57 mice. When the tumor grew to 500-800 mm³, the mice were divided into two groups based on tumor size. The concentration of ITGB1⁻mscIL12⁺HN3⁺Deg EV was adjusted to 2.5E+11 particles/ml and 200 μl was injected via the tail vein. After 30 min, the tumors from the mice were collected to assess the number of EV particles within the tumors.

(6) Antitumor effects of ITGB1⁻mscIL12⁺HN3⁺Deg EV in the Hepa1-6-hGPC3 subcutaneous tumor model mouse: Treatment was administered on day 4 and day 7 after tumor cell inoculation. The EV group received 5E+10 particles per mouse, while rmIL12 was administered at a dose of 0.7 ng per mouse, with 3 μg per mouse via tail vein injection. Tumor volume and weight were measured every two days during the treatment period. At the end of the experiment, the tumors from the mice were collected for pathological sectioning.

### 2. Results:

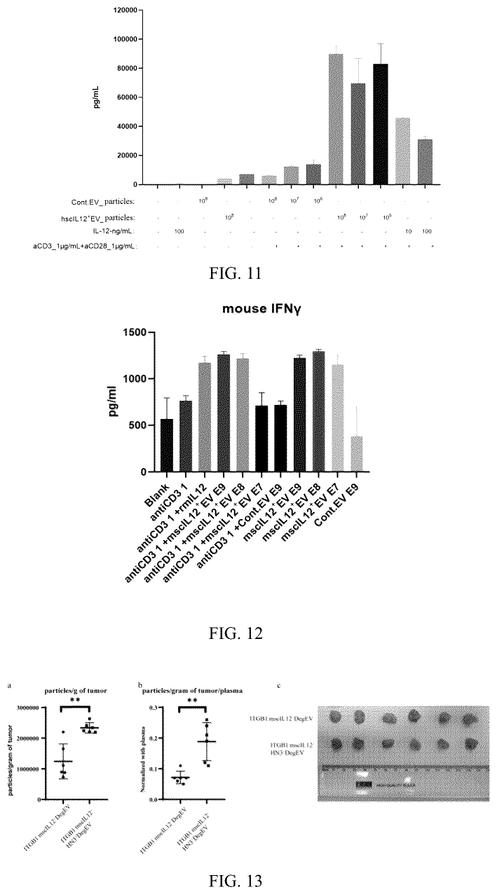

As shown in FIGS. 10-15 and Tables 8-13, the targeting capability of ITGB1⁻mscIL12⁺HN3⁺EV (or referred to as HN3⁺EV) was verified at the cellular level. Immunofluorescence detection: In the figures, the blue represented the cell nuclei, while the green indicated PKH67-labeled EVs. The results demonstrated that the EVs loaded with HN3 bound to the surface GPC3 of HepG2 cells, forming a green halo around the cell membrane. In contrast, the control EVs (without HN3) showed weak binding to the cells.

Cell binding assay: After pre-treating with hGPC3 protein, the EVs loaded with HN3 exhibited binding to free hGPC3 at the HN3 site. Consequently, the binding to cells expressing hGPC3 was significantly lower compared to the control EV group, while no significant differences were observed with cells that did not express hGPC3, indicating that HN3⁺EV could specifically bind to hGPC3 on the cell surface.

IFNγ secretion by hscIL12⁺EV-stimulated human PBMCs: Compared to control EVs (not loaded with hscIL12, cont.EV), EVs loaded with hscIL12 activated human PBMCs, stimulating them to secrete IFNγ. The activation exhibited a clear dose-dependent effect; as the number of EV particles increased, the activation of PBMCs intensified. The activation by 10⁶ hscIL12⁺EV was stronger than that by 10 ng/ml (2.4 ng) hscIL12 protein.

IFNγ secretion by mscIL12⁺EV-stimulated mouse splenocytes: Compared to the control EVs (ContEV) not loaded with mscIL12, EVs loaded with mscIL12 stimulated splenocytes to secrete IFNγ, regardless of whether the splenocytes were pre-activated with anti-CD3 antibody.

Targeting of ITGB1⁻mscIL12⁺HN3⁺Deg EV (or referred to as HN3⁺EV) in the Hepa1-6-hGPC3 subcutaneous tumor model mouse: The results showed that HN3⁺EV had a significantly higher concentration of particles per gram of tumor and a higher organ index (particle concentration per gram of tumor/plasma particle concentration) in the mouse Hepa1-6-hGPC3 subcutaneous tumor model compared to the non-HN3 loaded EVs, indicating that loading HN3 significantly enhanced the targeting ability of the EVs.

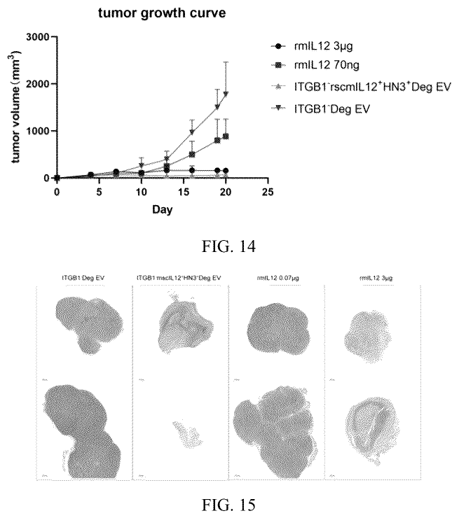

Antitumor effects of ITGB1⁻mscIL12⁺HN3⁺Deg EV in the Hepa1-6-hGPC3 subcutaneous tumor model mouse: The results showed that ITGB1⁻mscIL12⁺HN3⁺Deg EV significantly slowed tumor growth and inhibited tumor progression (as shown in the tumor growth curves in FIG. 14). The antitumor effect was superior to that of the 3 μg IL12 protein group. The HE staining results of the tumors revealed severe liquefaction in the rmIL12 3 μg group and the ITGB1⁻mscIL12⁺HN3⁺Deg EV group, while the rmIL12 70 ng group and the ITGB1 Deg EV group exhibited necrosis only at the center due to a lack of nutrients and oxygen (as shown in the tumor HE staining in FIG. 15).

TABLE 8
Binding of ITGB1⁻mscIL12⁺HN3⁺ EV to different cells

| HepG2 | | 293T-hGPC3 | | 293T | | Hepa1-6-hGPC3 | | Hepa1-6-mGPC3ko | |
|---|---|---|---|---|---|---|---|---|---|
| HN3⁺ | HN3⁺EV+ | HN3⁺ | HN3⁺EV+ | HN3⁺ | HN3⁺EV+ | HN3⁺ | HN3⁺EV+ | HN3⁺ | HN3⁺EV+ |
| EV | hGPC3 | EV | hGPC3 | EV | hGPC3 | EV | hGPC3 | EV | hGPC3 |
| 1.00 | 0.66 | 0.97 | 0.69 | 1.09 | 0.89 | 1.01 | 0.44 | 0.89 | 0.715 |
| 1.00 | 0.62 | 1.04 | 0.59 | 0.97 | 0.91 | 1.01 | 0.45 | 0.929 | 1.29 |
| 0.99 | 0.71 | 0.99 | 0.7 | 0.92 | 1.05 | 1.00 | 0.37 | 1.095 | 1.005 |
| 1.00 | 0.54 | | | 1.02 | 0.82 | | | 0.988 | 0.941 |
| | | | | | | | | 1.007 | 0.959 |
| | | | | | | | | 1.091 | 1.003 |

TABLE 9
hscIL12⁺EV stimulating human PBMCs to secrete IFNγ

| Cont. | hscIL12⁺ | IL12_ | antiCD3Ab | | | |
|---|---|---|---|---|---|---|
| EV_ | EV_ | ng/ | +antiCD28 | Detection | Detection | Detection |
| particle | particle | ml | Ab | well 1 | well 2 | well 3 |
| − | − | − | − | −3 | −3 | −3 |
| − | − | 100 | − | 343 | 52 | 79 |
| 1E+8 | − | − | − | −0.3 | 6.2 | 0.0 |
| − | 1E+8 | − | − | 6297 | 3449 | 2800 |
| − | − | − | + | 7587 | 7039 | 6682 |
| 1E+8 | − | − | + | 6427 | 5240 | 6481 |
| 1E+7 | − | − | + | 11351 | 12272 | 13323 |
| 1E+6 | − | − | + | 12469 | 17156 | 11964 |
| − | 1E+8 | − | + | 92760 | 83097 | 93880 |
| − | 1E+7 | − | + | 89634 | 58829 | 60023 |
| − | 1E+6 | − | + | 77268 | 73063 | 98684 |
| − | − | 10 | + | 47189 | 45026 | 45463 |
| − | − | 100 | + | 30343 | 33611 | 28886 |

TABLE 10
mscIL12⁺EV stimulating mouse splenocytes to secrete IFNγ

| | | antiCD3 + rmIL | antiCD3 + mscIL12⁺ | antiCD + mscIL12⁺ | antiCD3 + mscIL12⁺ |
|---|---|---|---|---|---|
| Blank | antiCD3 | 12 | EV E9 | EV E8 | EV E7 |
| 313.4 | 820.32 | 1226.79 | 1287.88 | 1173.99 | 662.74 |
| 640.15 | 713.63 | 1203.28 | 1231.7 | 1206.21 | 601.48 |
| 749.31 | 763.08 | 1098.37 | 1275.03 | 1279.97 | 867.76 |

| antiCD3 + Cont. | mscIL12⁺EV | mscIL12⁺EV | mscIL12⁺EV | |
|---|---|---|---|---|
| EV E9 | E9 | E8 | E7 | Cont. EV E9 |
| 716.37 | 1189.6 | 1300.74 | 1093.55 | 752.98 |
| 764 | 1249.4 | 1316.61 | 1091.63 | 223.52 |
| 682.69 | 1230.72 | 1277 | 1279.97 | 177.45 |

TABLE 11
EV particle concentration per gram of tumor tissue

| ITGB1⁻mscIL12⁺Deg EV | ITGB1⁻mscIL12⁺HN3⁺Deg EV |
|---|---|
| 895000 | 2410000 |
| 877000 | 2630000 |
| 2200000 | 2340000 |
| 1650000 | 2190000 |
| 732000 | 2200000 |
| 1110000 | 2240000 |

TABLE 12
Ratio of EV particle concentration per gram of tumor
tissue to plasma EV particle concentration

| ITGB1^{—}mscIL12⁺Deg EV | ITGB1^{—}mscIL12⁺HN3⁺Deg EV |
|---|---|
| 0.05 | 0.24 |
| 0.06 | 0.26 |
| 0.11 | 0.19 |
| 0.07 | 0.21 |
| 0.07 | 0.12 |
| 0.07 | 0.11 |

TABLE 13
Tumor growth data

| | Tumor volume (mm³) | | | | | | |
|---|---|---|---|---|---|---|---|
| Group | Day 4 | Day 7 | Day 10 | Day 13 | Day 16 | Day 19 | Day 20 |
| rmIL12 3 μg/mouse | 72 | 221 | 229 | 386.9 | 359 | 231 | 212 |
| | 40 | 102 | 98 | 184.2 | 203 | 209 | 190 |
| | 86 | 178 | 96 | 153.1 | 122 | 153 | 102 |
| | 164 | 176 | 95 | 96.5 | 137 | 205 | 216 |
| | 59 | 98 | 113 | 122.9 | 112 | 93 | 119 |
| | 0 | 127 | 77 | 107.2 | 108 | 118 | 113 |
| | 54 | 74 | 69 | 86.4 | 94 | 125 | 135 |
| rmIL12 70 ng/mouse | 63 | 64 | 65 | 122.7 | 357 | 489 | 726 |
| | 69 | 128 | 127 | 237.4 | 485 | 707 | 954 |
| | 67 | 74 | 83 | 267.2 | 396 | 704 | 791 |
| | 58 | 73 | 65 | 110.5 | 187 | 393 | 529 |
| | 61 | 76 | 86 | 186.4 | 411 | 651 | 547 |
| | 31 | 85 | 155 | 320.0 | 573 | 895 | 1045 |
| | 0 | 131 | 134 | 519.5 | 1080 | 1751 | 1599 |
| ITGB1⁻mscIL12⁺HN3⁺DegEV | 47 | 91 | 70 | 73.6 | 102 | 112 | 112 |
| | 62 | 77 | 81 | 40.2 | 42 | 45 | 65 |
| | 0 | 0 | 0 | 0.0 | 0 | 0 | 0 |
| | 197 | 86 | 95 | 83.3 | 85 | 140 | 192 |
| | 77 | 54 | 70 | 69.1 | 68 | 100 | 110 |
| | 0 | 61 | 64 | 0.0 | 0 | 0 | 0 |
| | 36 | 72 | 49 | 63.8 | 58 | 25 | 26 |
| ITGB1⁻Deg EV | 0 | 97 | 233 | 510.0 | 904 | 1667 | 1636 |
| | 0 | 113 | 134 | 178.3 | 1020 | 1608 | 832 |
| | 61 | 87 | 194 | 276.7 | 749 | 1094 | 1947 |
| | 94 | 71 | 103 | 229.8 | 521 | 840 | 1020 |
| | 91 | 128 | 120 | 358.8 | 1027 | 1936 | 2571 |
| | 0 | — | 480 | 634.0 | 1219 | 1731 | 2618 |
| | 0 | 131 | 534 | 578.0 | 1313 | 1609 | 1797 |

1. A method for preparing an extracellular vesicle (EV) with reduced non-specific binding/endocytosis, comprising steps of:
(1) knocking out ITGB1 in HEK293F cells at the cellular level to achieve ITGB1 knockout at the EV level,
(2) sorting and verifying an ITGB1 knockout HEK293F cell line,
(3) loading NanoLuc® into EVs from the ITGB1 knockout HEK293F cell line,
(4) verifying changes in binding/endocytosis function of EVs from ITGB1 knockout HEK293F cell line at the cellular level, and
(5) verifying the effect of ITGB1 knockout on the tissue distribution of EVs in major organs in vivo.

2. The method according to claim 1, wherein step (1) comprises transiently transfecting ITGB1 CRISPR/Cas9/gRNA plasmid into HEK293F suspension cells using PEI transfection reagent, incubating the cells with APC anti-human ITGB1 antibody after 72 h, sorting out APC-negative cell population by flow cytometry, and expanding the sorted cell population, resulting in an ITGB1 knockout HEK293F cell line.

3. The method according to claim 2, wherein step (2) further comprises identifying changes in ITGB1 expression on ITGB1 EVs by Western Blot.

4. The method according to claim 3, wherein step (3) comprises constructing pFc-TMD (NPTN-TMD)-ICD (EWI-F-ICD)-NanoLuc® plasmid using VB220306-1137jmq as a vector, co-transfecting the plasmid into the cells with Sleeping Beauty transposase expression vector pCMV-(CAT) T7-SB100X, adding Zeocin™ antibiotic at a final concentration of 100 μg/ml after 72 h until all cells in a non-stably transfected group die, expanding the cells, centrifuging cells at 5000 rpm for 30 min to collect supernatant, purifying EVs from the supernatant, establishing a standard curve of EV particle concentration-NanoLuc® enzyme activity to verify NanoLuc® activity in EVs, and treating EVs with Triton™ X-100 and Proteinase K to verify the loading position of NanoLuc® in EVs.

5. The method according to claim 4, wherein step (4) comprises inoculating recipient cells at 0.5E+5 cells per well into a 24-well plate, adding 50 μL of NanoLuc® detection substrate after treatment, and measuring NanoLuc® activity.

6. The method according to claim 5, wherein step (5) comprises injecting EVs via a tail vein of a mouse at a dose of 5E+10 particles, collecting plasma from peripheral blood at 5, 15, 30, 60, 120 and 240 min, homogenizing liver, spleen, lung and kidney tissues to obtain tissue homogenates, measuring NanoLuc® enzyme activity in the plasma and tissue homogenates, establishing a standard curve of EV particle concentration-NanoLuc® enzyme activity, and calculating EV particle concentration in the tissues.

7. The method according to claim 1, wherein the method further comprises a step (6) of subjecting EVs from the ITGB1 knockout cells to deglycosylation treatment.

8. The method according to claim 7, wherein step (6) comprises adding 10 μl of PNGase F and 100 μl of 10× Reaction Buffer per 2E+10 EVs to EVs from the ITGB1 knockout cells, supplementing the system with phosphate buffered saline (PBS) to 1 ml with EV concentration of 2E+10 p/ml, performing incubation at 37 °C for 1 h, and detecting CD63 and GAPDH by Western Blot.

9. The method according to claim 8, wherein verification of changes in binding/endocytosis of EVs with recipient cells after deglycosylation at the cellular level comprises adding NanoLuc® detection substrate and measuring NanoLuc® activity.

10. The method according to claim 8, wherein verification of the effect of deglycosylation on the tissue distribution of EVs in major organs in vivo comprises injecting EVs via a tail vein of a mouse at a dose of 5E+10 particles, collecting plasma from peripheral blood at 5, 15, 30, 60, 120 and 240 min, homogenizing liver, spleen, lung and kidney tissues to obtain tissue homogenates, measuring NanoLuc® enzyme activity in the plasma and tissue homogenates, establishing a standard curve of EV particle concentration-NanoLuc® enzyme activity, and calculating EV particle concentration in the tissues.

11. (canceled)

12. The method according to claim 1, where hod further comprises a step (7) of loading an antibody into EVs to confer targeting function, constructing plgkappa-HA-HN3-TMD (PDGFRB) plasmid using pHEK293_Ultra_Expression_I (Takara) as a vector, constructing a lentivirus or a transfection system of pLenti-HN3-Fc-ITGB1_TMD-ITGB1_ICD-PGK-Blasticidin S-WPRE, and stably infecting or transfecting into the ITGB1 knockout HEK293F cells.

13. (canceled)

14. The method according to claim 12, wherein the method further comprises a step (8) of loading scIL12 into the EVs to activate the immune system, constructing pIL12-Fc-TMD (NPTN-TMD)-ICD (EWI-F-ICD)-NanoLuc® plasmid for hscIL12 and mscIL12 using VB220306-1137jmq as a vector, co-transfecting the plasmid into the cells with Sleeping Beauty transposase expression vector pCMV-(CAT) T7-SB100X, co-infecting/transfecting into the ITGB1 knockout HEK293F cells with HN3 lentivirus and IL12 sleeping beauty plasmid, further screening and centrifuging cells to collect supernatant, purifying ITGB1⁻scIL12⁺HN3⁺EV from the supernatant, and further performing deglycosylation to obtain ITGB1⁻scIL12⁺HN3⁺Deg EV.

15. The method according to claim 14, wherein step (8) comprises using HepG2 cells with high expression of human GPC3 protein for immunofluorescence verification, and using HepG2 cells, Hepa1-6, HEK293T, and modified Hepa1-6-hGPC3 and HEK293T-hGPC3 cells with high expression of human GPC3 protein for cell binding verification, to verify the targeting effect of EVs.

16. The method according to claim 14, wherein the method further comprises treating human PBMCs with CD28 antibody, CD3 antibody and various concentrations of control EVs and measuring the expression of IFNγ stimulated by ITGB1⁻hscIL12⁺EV in human PBMCs in vitro by ELISA.

17. The method according to claim 14, wherein the method further comprises treating mouse splenocytes with CD3 antibody and various concentrations of control EVs and measuring the expression of IFNγ stimulated by ITGB1⁻scIL12⁺HN3⁺EV in mouse splenocytes in vitro by ELISA.

18. The method according to claim 14, wherein the method further comprises establishing an Hepa1-6-hGPC3 subcutaneous tumor model mouse and injecting ITGB1⁻mscIL12⁺HN3⁺Deg EV via a tail vein of the mouse to verify the targeting effect of ITGB1⁻mscIL12⁺HN3⁺Deg EV (or referred to as HN3⁺EV) in the Hepa1-6-hGPC3 subcutaneous tumor model mouse.

19. The method according to claim 18, wherein the method further comprises inoculating tumor cells, performing administration on day 4 and day 7, measuring tumor volume and weight every two days, weighting tumors, and collecting tumors for pathological sectioning, to verify the tumor inhibitory effect of ITGB1⁻mscIL12⁺HN3⁺Deg EV in the Hepa1-6-hGPC3 subcutaneous tumor model mouse.

20. (canceled)

21. (canceled)

22. (canceled)

23. A pharmaceutical composition comprising an extracellular vesicle (EV) with reduced non-specific binding/endocytosis, wherein the EV is prepared by the steps of:
(1) knocking out ITGB1 in HEK293F cells at the cellular level to achieve ITGB1 knockout at the EV level,
(2) sorting and verifying an ITGB1 knockout HEK293F cell line,
(3) loading NanoLuc® into EVs from the ITGB1 knockout HEK293F cell line,
(4) verifying changes in bindi is function of EVs from ITGB1 knockout HEK293F cell line at the cellular level, and
(5) verifying effect of ITGB1 knockout on the tissue distribution of EVs in major organs in vivo.

24. A method for treating hepatocellular carcinoma in a subject in need thereof, the method comprising administering a pharmaceutical composition to the subject con rising an extracellular vesicle (EV) with reduced non-specific binding/endocytosis, wherein the EV is prepared by a method comprising the steps of:
(1) knocking out ITGB1 in HEK293F cells at the cellular level to achieve ITGB1 knockout at the EV level,
(2) sorting and verifying an ITGB1 knockout HEK293F cell line,
(3) loading Nan Luc® into EVs rom the ITGB1 knockout HEK293F cell line,
(4) verifying changes in binding/endocytosis function of EVs from ITGB1 knockout HEK293F cell line at the cellular level, and
(5) verifying the effect of ITGB1 knockout on the tissue distribution of EVs in major organs in vivo.

25. The pharmaceutical composition according to claim 23, wherein the pharmaceutical composition is useful in the treatment of hepatocellular carcinoma with high expression of GPC3.


SEQUENCE LISTING (10 sequences)

SEQ ID NO: 1 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - protein, 168 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCER

SEQ ID NO: 3 - protein, 214 aa, synthetic construct
MCQSRYLLFLATLALLNHLSLARVIPVSGPARCLSQSRNLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 4 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 5 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 707 aa, synthetic construct
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGP

SEQ ID NO: 8 - protein, 118 aa, synthetic construct
MQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSS

SEQ ID NO: 9 - protein, 21 aa, synthetic construct
LWPFLGILAEIIILVVIIVVY

SEQ ID NO: 10 - protein, 26 aa, synthetic construct
SSHWCCKKEVQETRRERRRLMSMEMD